(19) United States
(12) Patent Application Publication
(10) Pub. No.: US 2008/0069854 A1
(43) Pub. Date: Mar. 20, 2008

(54) MEDICAL DEVICES AND METHODS OF MAKING AND USING

(75) Inventors: T. Danny Xiao, Willington, CT (US); MICHAEL DRUES, GRAFTON, MA (US); MARK ETTLINGER, LEXINGTON, MA (US); XINQING MA, WILLINGTON, CT (US)

Correspondence Address: CANTOR COLBURN, LLP, 20 Church Street, 22nd Floor, Hartford, CT, 06103, US

(73) Assignee: INFRAMAT CORPORATION, FRAMINGTON, CT (US)

(21) Appl. No.: 11/833,147

(22) Filed: Aug. 2, 2007

Related U.S. Application Data
(60) Provisional application No. 60/821,257, filed on Aug. 2, 2006.

(51) Int. Cl. A61F 2/00

(52) U.S. Cl. 424/423

(57) ABSTRACT

Disclosed herein are medical devices. The medical devices generally include a biocompatible nanostructured ceramic material having an average grain size dimension of about 1 nanometer to about 1000 nanometers, a strain to failure of at least about 1 percent, and a cross-sectional hardness greater than or equal to about 350 kilograms per square millimeter. Also disclosed are methods of making and using the medical devices.

### CROSS REFERENCE TO RELATED APPLICATION

This application claims the benefit of U.S. Provisional Patent Application No. 60/821,257 filed Aug. 2, 2006, which is incorporated by reference herein in its entirety.

### TECHNICAL FIELD

The present disclosure generally relates to medical devices and more specifically to medical devices comprising biocompatible nanoscale ceramic compositions.

### BACKGROUND

Surgical implantation of medical devices can structurally compensate for diseased, damaged, or missing musculoskeletal components, vascular system components, organs, and the like. Although some medical devices can last a few decades, a significant number fail much earlier, in part because of biocompatibility issues. As part of the body's immunological response to a recognized foreign body, many implanted medical devices experience a biofouling process called fibrous encapsulation in which local cells surround the implant and essentially wall off the implant from the body. Fibrous encapsulation and other biofouling processes are problematic for devices intended to interact with the body. For example, osseointegration of an orthopedic implant could be hindered or even prevented, drug delivery devices or biosensors could be rendered ineffective, and restenosis could occur in stented arteries or other such lumens.

In order to increase their service life and effectiveness, medical devices have been designed or fabricated using materials possessing surface properties that minimize biofouling at the tissue-device interface. For example, stainless steel has frequently been used as an implant material owing to the relatively passive oxide layer that forms on its surface. Alternatively, a coating composition, such as hydroxyapatite or a polymer, can be deposited on the surface of the implant to mask certain undesirable or less biofriendly properties of the underlying implant material. In other cases, a locally deliverable (i.e., to the area surrounding the implant) biologically active agent can be deposited oil the surface of the implant to minimize the body's response to the presence of the implant and/or to any injury caused by the implant during the implantation procedure.

### BRIEF SUMMARY

Disclosed herein are medical devices. In one embodiment, a medical device includes a biocompatible nanostructured ceramic material having an average grain size dimension of about 1 nanometer to about 1000 nanometers, a strain to failure of at least about 1 percent, and a cross-sectional hardness greater than or equal to about 350 kilograms per square millimeter.

In another embodiment, the medical device includes: a structural member comprising a metal, alloy, polymer, biologic scaffolding, or combination comprising at least one of the foregoing; and a film comprising a biocompatible nanostructured ceramic material at least partially coating a surface of the medical device, the film having an average grain size dimension of about 1 nanometer to about 1000 nanometers, a strain to failure of at least about 1 percent, and a cross-sectional hardness greater than or equal to about 350 kilograms per square millimeter.

In an embodiment, a method includes surgically implanting a medical device, comprising a biocompatible nanostructured ceramic material and having an average grain size dimension of about 1 nanometer to about 1000 nanometers, a strain to failure of at least about 1 percent, and a cross-sectional hardness greater than or equal to about 350 kilograms per square millimeter.

In one embodiment, a method of making a medical device includes consolidating a biocompatible nanoparticulate ceramic powder into a free standing bulk biocompatible ceramic nanostructured ceramic material having an average grain size dimension of about 1 nanometer to about 1000 nanometers, a strain to failure of at least about 1 percent, and a cross-sectional hardness greater than or equal to about 350 kilograms per square millimeter.

In another embodiment, a method of making a medical device includes disposing a coating of a biocompatible nanostructured ceramic material having an average grain size dimension of about 1 nanometer to about 1000 nanometers, a strain to failure of at least about 1 percent, and a cross-sectional hardness greater than or equal to about 350 kilograms per square millimeter onto at least a portion of a surface of a structural member of the medical device.

The above described and other features are exemplified by the following figures and detailed description.

### BRIEF DESCRIPTION OF THE DRAWINGS

Referring now to the figures, which are exemplary embodiments and wherein like elements are numbered alike:

FIG. 1 schematically illustrates a cross section of a medical device having a dense, free standing bulk biocompatible nanostructured ceramic member;

FIG. 2 schematically illustrates a cross section of a medical device having a porous., free standing bulk biocompatible nanostructured ceramic member;

FIG. 3 schematically illustrates a cross section of a medical device having a dense, biocompatible nanostructured ceramic coating disposed on a surface of a structural member of the medical device;

FIG. 4 schematically illustrates a cross section of a medical device having a porous, biocompatible nanostructured ceramic coating disposed on a surface of a structural member of the medical device;

FIGS. 5(a) and (b) schematically illustrate a cross section of a medical device having a tissue adherent material and a biocompatible nanostructured ceramic coating disposed on a structural member of the medical device;

FIG. 6 schematically illustrates a cross section of a medical device having a biocompatible nanostructured ceramic coating disposed on a tissue adherent material or a metal layer; and

FIG. 7 schematically illustrates a cross section of a medical device having a biocompatible nanostructured ceramic coating and a tissue adherent material or a metal layer disposed on opposing surfaces of a structural member of the medical device.

### DETAILED DESCRIPTION

Medical devices and methods of making and using the devices are described herein. The medical devices are devices that can be surgically implanted and generally include a biocompatible nanostructured ceramic material. Nanostructured materials can have superior properties compared to those with larger grain sizes including improved toughness, hardness, wear resistance, and/or ductility. In an advantageous feature the medical devices disclosed herein experience minimal or no biofouling and thus exhibit improved biocompatibility compared with currently available medical devices.

As used herein. “biocompatible” refers to a material that, when placed in contact with a body, does not cause the body to attack or reject it. As used herein, “nanostructured” generally refers a material having an average grain size dimension of about 1 nanometer (nm) to about 1000 nm. In one embodiment, the average grain size dimension of the biocompatible nanostructured ceramic material is less than or equal to about 500 nm. In another embodiment, the average grain size dimension of the biocompatible nanostructured ceramic material is less than or equal to about 250 nm. In yet another embodiment, the average grain size dimension of the biocompatible nanostructured ceramic material is less than or equal to about 100 nm. In still another embodiment, the average grain size dimension of the biocompatible nanostructured ceramic material is greater than or equal to about 10 nm. In still another embodiment, the average grain size dimension of the biocompatible nanostructured ceramic material is greater than or equal to about 25 nm.

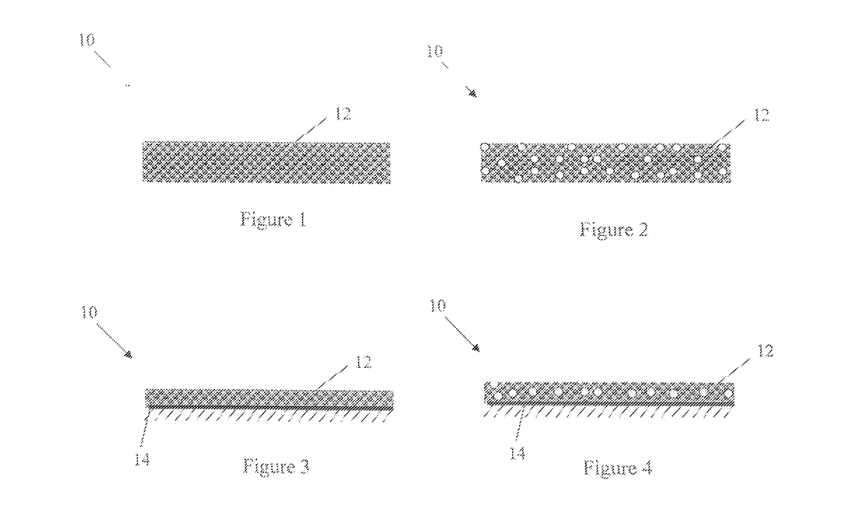

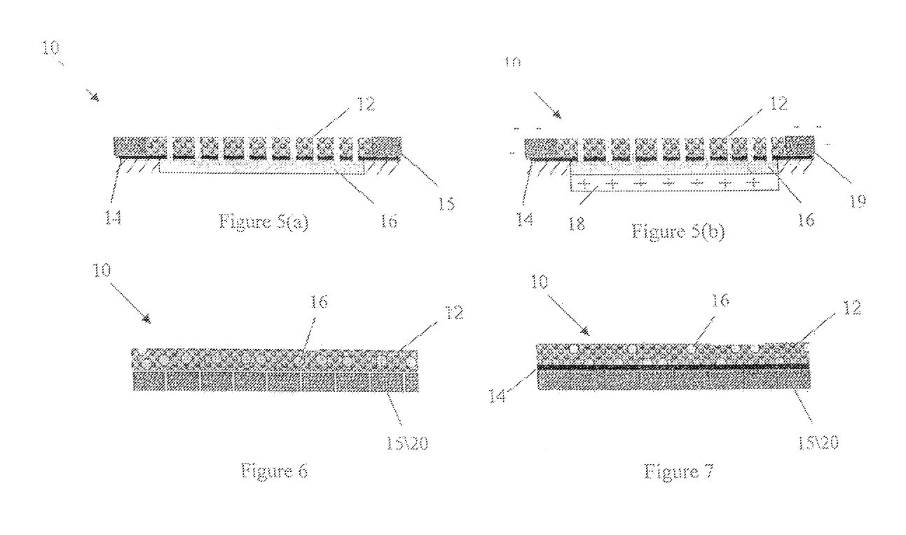

Referring now to FIGS. 1 through 4, wherein cross sections of exemplary medical devices, generally designated by the numeral 10, are shown. The nanostructured ceramic material, generally designated by the numeral 12, can take the form of a free standing bulk member, as illustrated in FIGS. 1 and 2. Alternatively, as shown in FIGS. 3 and 4, the nanostructured ceramic material 12 can be a layer that is coated onto a surface of a structural member 14 of the medical device 10. Further, the nanostructured ceramic material 12 can be highly dense (i.e., greater than or equal to about 90% dense, based on the theoretical density of the nanostructured ceramic material 12) as shown in FIGS. 1 and 3; or the nanostructured ceramic material 12 can be porous (i.e., greater than or equal to about 10% porous, based on the total volume of the nanostructured ceramic material 12), as shown in FIGS. 2 and 4. The particular form of the nanostructured ceramic material 12 and/or its density/porosity call be determined by the specific type of medical device 10 used, as will be discussed in more detail hereinbelow.

Suitable ceramic compositions for use in the medical device 10 include, but are not limited to, hard phase oxides such as Al₂O₃, Cr₂O₃, ZrO₂, TiO₂, SiO₂. Y₂O₃, CeO₂, and the like; metal carbides such as Cr₃C₂, WC, TiC, ZrC, B₄C, and the like; diamond; metal nitrides such as cubic BN, TiN, ZrN, HfN, Si₃N₄, AlN, and the like; metal borides such as TiB₂, ZrB₂, LaB, LaB₆, W₂B₂, AlB₂, and the like; and combinations comprising at least one of-the foregoing compositions. The wear characteristics of hard phase metal oxides, carbides, nitrides, and borides are superior to biomimetic materials such as hydroxyapatite and other phosphate-based materials.

In one embodiment, the biocompatible nanostructured ceramic material 12 is a composite comprising at least 51 volume (vol) %, based oil the total volume of the composite, of a nanostructured ceramic composition; and a nanostructured binder phase composition comprising a relatively soft and low melting ceramic material. The concentration of the binder phase can be, for example, about 0 weight (wt) % to about 50 wt %, based on the total weight of the composite. Suitable ceramic binder phase compositions for the composite include, but are not limited to, SiO₂, CeO₂, Y₂O₃, TiiO₂, and combinations comprising at least one of the foregoing ceramic binder phase compositions.

In another embodiment, the biocompatible nanostructured ceramic material 12 is a composite of a nanostructured ceramic composition and a nanostructured metal composition, i.e., a “cermet”. The concentration of the metal composition can be, for example, about 0 wt % to about 50 wt %, based on the total weight of the composite. Suitable cermets include, but are not limited to, WC/Co, TiC/Ni, TiC/Fe, Ni(Cr)/Cr₃C₂, WC/CoCr, and combinations comprising at least one of tile foregoing. Tile cermet can further include a grain growth inhibitor such as TiC, VC, TaC, and HfC, or other additives such as Cr, Ni, B, and BN.

In still another embodiment, the biocompatible nanostructured ceramic material 12 can be a combination comprising at least one of the foregoing ceramics, ceramic composites, or cermets.

The substrate (i.e., the structural member 14), for those embodiments in which the biocompatible nanostructured ceramic material 12 is a coating, can be formed from a metal, alloy, polymer, biologic scaffolding, or a combination comprising at least one of the foregoing. The thickness of the substrate can vary depending on the use of the medical device. For example, the thickness of the substrate can be selected to ensure that is sufficiently flexible or ductile to promote adhesion of the coating. The relatively corrosive environment combined with the low tolerance of the body for even minute concentrations of various metallic corrosion products eliminates from discussion many metals. Of the metallic candidates that have tile required mechanical strength and biocompatibility, stainless steel alloys such as type 316 L, chromium-cobalt-molybdenum alloys titanium alloys such as Ti₆Al₄V, zirconium alloys, shape memory nickel-titanium alloys, super elastic nickel-titanium alloys, and combinations comprising at least one of the foregoing alloys have proven suitable for use as structural members 14. These materials can be shaped into the desired form of the medical device by, for example, casting, machining, forging, extruding, drawing(sheet & wire), deep drawing, and rapid or direct fabrication methods such as SLS (stereo laser sintering), FMD (fused metal deposition), DMLS (direct metal laser sintering). Post fabrication processes can include conventional machining such as milling, lathing, and grinding and unconventional machining such as EDM wire & sinker, laser cutting, chemical machining, waterjetting, laser, plasma, arc, and friction welding, photochemical processes such as etching, physical or chemical vapor deposition, and composite bonding methods.

The polymers used to form the structural component 14 can be biodegradable, non-biodegradable, or combinations thereof. In addition, fiber- and/or particle-reinforced polymers can also be used. Non-limiting examples of suitable non-biodegradable polymers include polyisobutylene copolymers and styrene-isobutylene-styrene block copolymers, such as styrene-isobutylene-styrene tert-block copolymers (SIBS); polyvinylpyrrolidone including cross-linked polyvinylpyrrolidone; polyvinyl alcohols; copolymers of vinyl monomers such as EVA; polyvinyl ethers; polyvinyl aromatics; polyethylene oxides; polyesters such as polyethylene terephthalate; polyamides; polyacrylamides; polyethers such as polyether sulfone; polyalkylenes such as polypropylene, polyethylene, highly crosslinked polyethylene, and high or ultra high molecular weight polyethylene; polyurethanes; polycarbonates; silicones; siloxane polymers; cellulosic polymers such as cellulose acetate; and combinations comprising at least one of the foregoing polymers.

Non-limiting examples of suitable biodegradable polymers include polycarboxylic acid; polyanhydrides such as maleic anhydride polymers; polyorthoesters; poly-amino acids; polyethylene oxide; polyphosphazenes; polylactic acid, polyglycolic acid, and copolymers and mixtures thereof such as poly(L-lactic acid) (PLLA), poly(D,L,-lactide), poly(lactic acid-co-glycolic acid), and 50/50 weight ratio (D,L-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-blutylacrylate; polyhydroxybutyrate valerate and mixtures thereof; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid, cellulose, and hydroxypropylmethyl cellulose; gelatin; starches; dextrans; and alginates and derivatives thereof, proteins and polypeptides; and mixtures and copolymers of any of the foregoing. The biodegradable polymer can also be a surface erodable polymer such as polyhydroxybutyrate and its copolymers, polycaprolactone, polyanhydrides (both crystalline and amorphous), and maleic anhydride.

If more than one surface of the structural member 14 of the medical device 10 comprises a biocompatible nanostructured ceramic material 12 coating, it is not necessary that each of the structural members 14 be formed from the same type of material. Nor is it necessary for a medical device 10 to have only one biocompatible nanostructured ceramic material 12 coating disposed on a structural member 14. For example, one coating can be disposed on a tissue or body-contacting portion of the structural element 14, while another coating can be disposed on a non-contacting portion of the structural element 14.

For medical devices 10, such as those whose cross sections are shown in FIGS. 1 and 2, the bulk nanostructured ceramic material 12 can be formed by consolidating a nanoparticulate ceramic powder into a free standing bulk member. Optionally, other ceramic and/or metal powders can be consolidated with that first ceramic powder to form a bulk composite member. The consolidation can be accomplished by sintering the powder, either under pressure or without pressure. Specific sintering processes include, but are not limited to, hot pressing, hot isostatic pressing (“hiping”), pressureless sintering at elevated temperatures, and the like. Alternatively, the nanoparticulate powder can be either extruded or injection molded into a desired shape. The consolidation parameters can be adjusted to obtain the desired level of density or porosity.

In one embodiment, the free standing bulk member can be formed by depositing a coating of the nanostructured ceramic material 12 onto a substrate, followed by post-deposition removal of the substrate from the coating. In this manner, the free standing bulk member can adopt the particular contours of the substrate without need for a separate shaping process. The depositing of the coating can be performed by, e.g., spin coating, casting, thermal spray, etc. The thickness of the bulk ceramic material 12 can vary depending on the intended use of the medical device 10. For example, the thickness can be greater than about 1 millimeter (mm). Examples of suitable substrates include, but are not limited to, metals, polymers such as biodegradable polymers, and composites comprising at least one of the foregoing. The removal of the substrate from the coating can be performed by, e.g., dissolving the substrate using an appropriate chemical, physical peel off, etc.

For medical devices 10, such as those whose cross sections are shown in FIGS. 3 and 4, the biocompatible nanostructured ceramic material 12 can be coated onto the surface of the structural member 14 by any known deposition method. Examples of suitable deposition methods include, but are not limited to, thermal spray, chemical vapor deposition, physical vapor deposition, sputtering, ion plating, cathodic arc deposition, atomic layer epitaxy, molecular beam epitaxy, powder sintering, electrophoresis, electroplating, injection molding, or the like. Thermal spray techniques involve deposition of materials in a molten or semi-molten state to form a coating on a substrate. Thermal spray can be performed using a powdered feedstock or a solution precursor. Examples of thermal spray techniques include plasma spray, dc-arc spray, high velocity oxygen fuel (HVOF) spray, laser thermal spray, and electron beam spray. For ceramic and ceramic composite coatings, plasma thermal spray is more favorable, while HVOF is more favorable for cermet-containing coating deposition.

In the HVOF spray process, nanometer-sized particles are desirably used as starting materials for reconstitution of a sprayable feedstock via a spray dry process. The substrate can optionally be prepared by degreasing and coarsening by sand blasting. As used herein, the term “substrate” refers to the structural member 14 of the medical device 10 that will be coated with the biocompatible nanostructured ceramic material 12 or a shaped article onto which a coating will be deposited and subsequently removed to form a free standing bulk member of the biocompatible nanostructured ceramic material 12. A high velocity flame is generated by combustion of a mixture of fuel (e.g., propylene) and oxygen. The enthalpy and temperature can be adjusted by using different fuels, different fuel-to-oxygen ratios, and/or different total fuel/oxygen flow rates. The nature of the flame can be adjusted according to the ratio of fuel to oxygen. Thus, an oxygen-rich, neutral or fuel-rich flame can be produced. The feedstock is fed into the flame at a controlled feed rate via, for example, a co-axial powder port, melted and impacted on the target substrate to form a deposit/film. The coating thickness can be controlled by the number of coating passes. The resultant coatings are optionally heat treated via an annealing step.

In the plasma spray process, nanometer-sized particles can be used as starting materials for the reconstitution of a sprayable feedstock via a spray dry process. Similarly, the substrate can optionally be prepared by degreasing and coarsening by sand blasting. A plasma arc is a source of heat that ionizes a gas, which melts the coating materials and propels it to the work piece. Suitable gases include argon, nitrogen, hydrogen, and the like. Plasma settings, which can be varied, include current voltage, working gases and their flow rates. Other process parameters include standoff distance, powder feed rate, and gun movements. One ordinarily skilled in the art in view of this disclosure could identify optimal conditions for each of the parameters without undue experimentation. Coating thickness can be controlled based on the number of coating passes. The resultant coatings are optionally heat treated via an annealing step.

Powdered feedstock can be prepared for thermal spray techniques including HVOF and plasma spray via the formation of micrometer-sized (e.g., 1 to 1000 micrometers (μm)) agglomerates containing individual nanoparticles (e.g., 1 to 1000 nanometers (nm) in size) and an insulating material. Individual nanoparticles can be difficult to thermally spray directly owing to their fine size and low mass. Agglomeration of the nanoparticles to form micrometer-sized granules allows for formation of a suitable feedstock. Formation of the feedstock can comprise dispersion (e.g., by ultrasound) of the nanoparticles into a liquid medium; addition of a binder to form a solution; spray drying of the solution into agglomerated particles; and heating the agglomerated particles to remove organic binders and to promote powder densification. Optionally, materials required to form a composite feedstock can also be dispersed in the liquid medium with the nanoparticles.

In organic-based liquid media, the binder can comprise about 5% to about 15% by weight, and preferably about 10% by weight, of paraffin dissolved in a suitable organic solvent. Suitable organic solvents include, for example, hexane, pentane, toluene and the like, and combinations comprising one or more of the foregoing solvents. In aqueous liquid media, the binder can comprise an emulsion of polyvinyl alcohol (PVA), polyvinylpyrrolidone (PVP), carboxymethyl cellulose (CMC), another water soluble polymer, or a combination comprising one or more of the foregoing polymers, formed in de-ionized water. The binder can be present in an amount of about 0.5% to about 5% by weight of the total aqueous solution, and preferably from about 1% to about 10% by weight of the total aqueous solution. In one embodiment, the binder is CMC.

A precursor solution can alternatively be prepared for the plasma spray process. The solution precursor can be fed into a plasma torch to deposit thick films up to several hundred micrometers and even several millimeters thick.

The precursor plasma spray process is described in more detail in commonly assigned U.S. Pat. No. 6,447,848, wherein this description is incorporated herein by reference. This process can entail the following steps: (1) preparing the precursor solution; (2) delivering the precursor solution using a solution delivery system; and (3) converting the precursor solution into a solid material by a pyrolysis reaction. The solution delivery system is used to drive the solution from a reservoir to a liquid injection nozzle that generates droplets with a size and velocity sufficient for their penetration into the core of a flame. The liquid flow rate and injection are controllable. Delivery of the solution typically comprises spraying of the solution into a chamber, onto the target substrate, or into a flame directed at the substrate. The substrate call be optionally heated. The resultant films can be optionally heat treated with an annealing procedure.

The precursor solution can be formed from at least one precursor salt dissolved in a solvent or a combination of solvents. Exemplary salts include, but are not limited to, carboxylate salts, acetate salts, nitrate salts, chloride salts, alkoxide salts, butoxide salts and the like, and combinations comprising one or more of the foregoing salts. The salts can be combined with alkali metals, alkaline earth metals, transition metals, rare earth metals, or tie like, and combinations comprising one or more of the foregoing metals. Precursors can also be in the form of inorganic silanes such as, for example, tetraethoxysilane (TEOS), tetramethoxysilane (TMOS), and the like, and combinations comprising one or more of the foregoing silanes. Exemplary solvents in which the salts can be dissolved include, but are not limited to, water, alcohols, acetone, methyl ethyl ketone, and combinations comprising one or more of the foregoing solvents. The reagents are weighed according to the desired stoichiometry of the final compound and then added and mixed into a liquid medium. The precursor solution can be heated and stirred to dissolve the solid components and to homogenize the solution.

The plasma spray can be performed in a manner suitable to produce a particular microstructures of the coating of the biocompatible nanostructured ceramic material 12. In one embodiment, the microstructure is a highly dense biocompatible nanostructured ceramic material 12, as seen in FIGS. 1 and 3, generally having a density greater than or equal to about 70% of the theoretical density. Theoretical density refers to the x-ray density or calculated density based on the weight and volume of each molecule for a given material. Specifically, the density of the biocompatible nanostructured ceramic material 12 is greater than or equal to about 95% of the theoretical density. More specifically, the density of the biocompatible nanostructured ceramic material 12 is greater than or equal to about 98% of the theoretical density. Even more specifically, the density of the coating is greater than or equal to about 99% of the theoretical density.

The solution plasma spray method employed to produce the dense microstructure can comprise injecting precursor solution droplets into a thermal spray flame, wherein a first portion of the precursor solution droplets are injected into a hot zone of the flame, and a second portion of the precursor solution droplets are injected into a cool zone of the flame; fragmenting the droplets of the first portion to form reduced size droplets and pyrolizing the reduced size droplets to form pyrolized particles in the hot zone; at least partially melting the pyrolized particles in the hot zone; depositing the at least partially melted pyrolized particles on the substrate; fragmenting at least part of the second portion of precursor solution droplets to form smaller droplets and forming non-liquid material from the smaller droplets; and depositing the non-liquid material on the substrate. The substrate can be optionally preheated and/or maintained at a desired temperature during deposition. As readily understood by one of ordinary skill in the art, the terms first portion and second portion do not imply a sequential order but are merely used to differentiate the two portions.

In another embodiment, the microstructure is a porous biocompatible nanostructured ceramic material 12, as seen in FIGS. 2 and 4, having a porosity generally greater than or equal to about 10% of the volume of the biocompatible nanostructured ceramic material 12. Specifically, the porosity of the biocompatible nanostructured ceramic material 12 is greater than or equal to about 15% of the volume of the biocompatible nanostructured ceramic material 12. More specifically, the porosity of the biocompatible nanostructured ceramic material 12 is greater than or equal to about 20% of the volume of the biocompatible nanostructured ceramic material 12. The porosity can be controlled by adjusting processing parameters such as green body formation and sintering temperature or by incorporating nonpermanent material in the coating process, followed by post-removal of the nonpermanent material.

Within the biocompatible nanostructured ceramic material 12, the existing pores generally have an average longest dimension less than or equal to about 1 μm. In one embodiment, the average longest dimension of the pores within the biocompatible nanostructured ceramic material 12 is less than or equal to about 500 nm. In another embodiment, the average longest dimension of the pores within the biocompatible nanostructured ceramic material 12 is less than or equal to about 100 nm. In yet another embodiment, the average longest dimension of the pores within the biocompatible nanostructured ceramic material 12 is less than or equal to about 10 nm.

Prior to coating the biocompatible nanostructured ceramic material 12 onto the particular structural member 14, a layer of the surface of the structural member 14 can be optionally oxidized. When the structural member 14 is metallic, this oxidized layer can serve as a corrosion barrier to prevent the metallic structural member 14 from undergoing corrosion and releasing metallic ions into the bloodstream. The oxidation can comprise preheating, electrolytic anodizing, passivating in a nitric acid bath, or the like.

Furthermore, after coating the biocompatible nanostructured ceramic material 12 onto the substrate (i.e., structural member 14 or a removable shaped article), and prior to characterization and/or implementation of the medical device 10, the coating can optionally be further processed, e.g., abraded, ground and/or polished to adjust a coefficient of friction and/or surface roughness, plasma treated, sterilized, or the like. Additional layers also call be added to provide additional functionality or desired characteristics to the coating as will be described in more detail below. However, in one specific embodiment, the coated structural member 14 is used as is, that is, without grinding or further processing. In still another specific embodiment, the as-deposited coating is abraded or polished as desired, but not further processed, e.g., not hydrated in order to enhance bonding between the coating and the substrate, not subjected to further coating, not consolidated, or the like. In such embodiments, the elimination of additional processing steps results in more economical manufacture of the medical devices 10.

The deposition processes described herein advantageously can form thicker and more uniform coatings, in the form of biocompatible nanostructured ceramic material 12, upon structural member 14. The coatings also adhere well to structural member 14 and can minimize friction during delivery of the medical device to which they are applied. Thus, the thickness of tile biocompatible nanostructured ceramic material 12 is generally greater than or equal to about 500 nm. In one embodiment, the average thickness of the biocompatible nanostructured ceramic material 12 is greater than or equal to about 1 μm. In another embodiment, the average thickness of the biocompatible nanostructured ceramic material 12 is greater than or equal to about 10 μm. In yet another embodiment, the thickness of the biocompatible nanostructured ceramic material 12 is less than or equal to about 1 millimeter (mm). Without intending to be limited by theory, it is postulated that a thicker biocompatible nanostructured ceramic material 12 (specifically greater than or equal to about 20 μm, and even more specifically greater than or equal to about 50 μm) advantageously provides increased hardness, increased fatigue resistance, increased ductility, and/or less grain pull out (i.e., particulate debris) during interaction between the medical device 10 and the body. This call result in implants with service lifetimes that can be significantly prolonged. For example, a coating having an average thickness greater than or equal to about 20 μm is expected to last longer than a coating having an average thickness greater than or equal to about 10 μm. In turn, a coating having an average thickness greater than or equal to about 50 μm is expected to last longer than a coating having an average thickness greater than or equal to about 20 μm. In a clinical setting, a practitioner accordingly might prefer use of a medical device having a coating with a thickness greater than or equal to about 50 μm over a medical device having a coating with a thickness greater than or equal to about 20 ρm, depending on the use of the medical device 10.

It should be recognized that if minimizing the overall thickness of the medical device 10 is desired, the use of thicker coatings of the biocompatible nanostructured ceramic material 12 can be compensated for by using a thinner structural member 14.

The biocompatible nanostructured ceramic material 12 can have a cross-sectional hardness (i.e., Vickers Hardness) greater than or equal to about 350 kilograms per square millimeter (kg/mm²). In one embodiment, the hardness of the biocompatible nanostructured ceramic material 12 is greater than or equal to about 500 kg/mm². In another embodiment, the hardness of the biocompatible nanostructured ceramic material 12 is greater than or equal to about 750 kg/mm². In yet another embodiment, the hardness of the biocompatible nanostructured ceramic material 12 is greater than or equal to about 1000 kg/mm². It is possible for the hardness of the biocompatible nanostructured ceramic material 12 to be up to about 8,000 kg/mm².

The biocompatible nanostructured ceramic material 12 can have a strain to failure (i.e., ductility) of greater than or equal to about 1 percent. In one embodiment, the strain to failure of the biocompatible nanostructured ceramic material 12 is greater than or equal to about 3 percent. In another embodiment, the strain to failure of the biocompatible nanostructured ceramic material 12 is greater than or equal to about 5 percent. In yet another embodiment, the strain to failure of the biocompatible nanostructured ceramic material 12 is greater than or equal to about 7 percent. It is possible for the strain to failure of the biocompatible nanostructured ceramic material 12 to be up to about 15 percent.

In certain embodiments, the medical device 10 can optionally include a “biologically active agent” (not shown) such as a “drug,” “therapeutic agent,” “pharmaceutically active material,” and “biologic”. These and other related terms in the art can be used interchangeably herein to generally refer to compositions that can be locally administered within the body of a patient at the implantation site to provide a biological effect. The biological effect can be, for example, a treatment of a diseased or abnormal condition, a preventive measure to inhibit future diseased or abnormal condition, a reduction in the body's response to the presence of the medical device 10, a reduction to an injury caused by the medical device 10 during the implantation procedure, or the like.

In various embodiments, the biologically active agent can be disposed directly upon, within the pores of, and/or underneath the biocompatible nanostructured ceramic material 12. In other embodiments, the biologically active agent can be dispersed in the ceramic material by co-deposition of the ceramic material and the biologically active agent or by mixing of the two together before depositing the mixture. If the biologically active agent is disposed underneath the biocompatible nanostructured ceramic material 12, it can pass through and/or around ceramic material 12 so that its therapeutic effect can be received. The concentration of the biologically active agent can vary depending on the intended use of the medical device 10.

In another embodiment, the biologically active agent can be incorporated into an optional polymeric coating (not shown) disposed on the medical device 10 or applied onto the optional polymeric coating. The polymers of the polymeric coatings can be biodegradable or non-biodegradable. Such polymers can include those polymers described above in addition to a polymer dispersion such as a polyurethane dispersion, a squalene emulsion, or a copolymer or mixture of any of the foregoing polymers.

In an embodiment in which the biologically active agent is deposited upon the medical device 10, it can be applied as a coating, alone, or in combination with solvents in which the therapeutic agent is at least partially soluble, dispersible, or emulsified, and/or in combination with polymeric materials as solutions, dispersions, suspensions, lattices, and the like. The solvents can be aqueous or non-aqueous. A coating comprising the biologically active material with solvents can be dried or cured, with or without added external heat, after being deposited on the medical device 10 to remove the solvent.

The biologically active agent can be any pharmaceutically active material such as a non-genetic therapeutic agent, a biomolecule, a small molecule, cells, a prophylactic agent, e.g., a vaccine, and the like. The biologically active agent can be disposed to provide for controlled release into the bloodstream, which includes long-term or sustained release.

Exemplary non-genetic therapeutic agents include, but are not limited to anti-thrombogenic agents such as heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonside, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl) ethyleneglycol-N,N,N′,N′-tetraacetic acid and in mixtures thereof; antibiotics such as gentamycin, rifampin, minocyclini, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate addicts, and polymeric or oligomeric NO addicts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, dicumarol, aspirin, prostaglandin inhibitors, platelet aggregation inhibitors such as cilostazol and tick antiplatelet factors; vascular cell growth promoters such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules comprising of a growth factor and a cytotoxin, bifunctional molecules comprising an antibody and a cytotoxin; cholesterol-lowering agents; vasodilating agents; agents which interfere with endogeneus vascoactive mechanisms; inhibitors of heat shock proteins such as geldanamycin; and any combinations comprising at least one of the foregoing.

Exemplary biomolecules include, but are not limited to, peptides, polypeptides and proteins; oligonucleotides; nucleic acids such as double or single stranded DNA (including naked and CDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids can be incorporated into delivery systems such as, for example, vectors (including viral vectors), plasmids or liposomes.

Non-limiting examples of proteins include, but are not limited to, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10 BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bc1-2 family factors and Akt kinase and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include, but are not limited to, hormones, nucleotides, amino acids, sugars, and lipids and compounds having a molecular weight of less than 100 kiloDaltons (kD).

Exemplary cells include, but are not limited to, stem cells, progenitor cells, endothelial cells, adult cardiomyocytes, and smooth muscle cells. Cells can be of human origin (autologous or allogenic) or from an animal source (xenogenic), or genetically engineered.

Any of the foregoing biologically active agents can be combined to the extent such combination is biologically compatible.

For some applications, a medical device surface is desired that can prohibit bio-fouling while it is desirable for an adjacent surface to provide an adhesive function. Thus, selective coatings can be applied to the varied surfaces of a medical device substrate to achieve the desired affects. FIG. 5(a) illustrates another embodiment of medical device 10 in which different coatings are formed upon the surface of structural member 14. As shown, on the side of the structural member 14 next to an organ, an adherent material 15 can be applied to particular areas, such as the edges of the structural member 14, to promote adhesion to a tissue. In the areas where drug delivery is preferred unencumbered by bio-fouling, the structural member 14 can be coated with a nanostructured ceramic material 12. A biologically active agent 16, e.g., a drug, can be disposed beneath structural member 14, which contains openings through which the agent 16 can pass. The biologically active agent 16 can be released by passing it into and exuding it through the pores of the nanostructured ceramic material 12. One application for this embodiment is an organ trans-tissue patch for drug delivery.

Examples of suitable adherent materials 15 include, but are not limited to, adhesive metals, alloys, polymers, biologic scaffolding, and combinations comprising at least one of the foregoing. Commercially available biocompatible adhesives and glues can be used. The adherent material 15 can be applied to the structural member 14 with or without a post process treatment that enhances adhesion to a tissue. Examples of such post process treatments include, but are not limited to, plasma etching, passivation or other acid etching, dimpling, bead blasting, and other modeled deformation means. The adherent material 15 can also be treated with coatings or solutions of organic or biologic chemistry that enhance adhesion.

FIG. 5(b) illustrates an embodiment similar to the one shown in FIG. 5(a) that utilizes iontophoresis for drug delivery. In this embodiment, the adherent material 15 is replaced by a cathode 18, and an anode 19 is formed beneath the biologicially active agent 16. Dissimilar metals can be used as the electrodes, i.e., cathode 18 and anode 19, to form a passive circuit for drug delivery. The biologically active agent 16, which resides in a reservoir beneath the structural member 14, can be dissolved in an aqueous solution to allow it to dissociate into positively charged cations and negatively charged anions. When a direct electric current is passed through this solution, the cations respond by moving toward the negative anode 19 and passing through perforations in the nanostructured ceramic material 12 to body tissue. In another embodiment, the cathode 18 and the anode 19 can be reversed to allow anions of the biologically active agent 16 to migrate to the ceramic material 12. Additional disclosure related to iontophoresis can be found in Tiwary et al. “Innovataions in Transdermal Drug Delivery; Formulations and Techniques.” Recent Patents on Drug Delivery & Formulation 2007: I, 23-26, wherein tile discussion related to iontophoresis is incorporated by reference herein.

FIG. 6 illustrates another embodiment of medical device 10 in which a biocompatible nanostructured ceramic material 12 is disposed upon an adherent material 15. The ceramic material 12 can be impregnated with a biologically active agent 16 that can exude from the ceramic material 12 to an adjacent tissue. Alternatively, the adherent material 15 can be replaced by or supplemented by a metal layer 20. Pure (unoxidized) precious metals have particular properties that can enhance or augment the function of the nanocomposite ceramics. These materials can form an antibacterial or antiviral barrier adjacent to the ceramic material 12 or provide some other metalobiologic function. Examples of precious metals include, but are not limited to, gold, silver, platinum, palladium, rhodium, and combinations comprising at least one of the foregoing metals. Some metals can be employed for topical, dermal, or surgical applications and for long term implant use. Examples of such metals include, but are not limited to, copper, zinc, nickel, cobalt, chromium, vanadium, zirconium, molybdenum, tin, silicon, aluminum, iron, other metals, and combinations comprising at least one of the foregoing meals. The effectiveness of these metals is improved as the purity of the metal is increased.

FIG. 7 illustrates yet another embodiment of medical device 10 in which a biocompatible nanostructured ceramic material 12 and an adherent material 15 or a metal layer 20 like those described above are disposed upon opposite sides of a structural member 14. The ceramic material 12 can be impregnated with a biologically active agent 16 that can exude from the ceramic material 12 to an adjacent tissue.

In the foregoing embodiments, the medical device 10 can be used in accordance with its general purpose as is known to one of ordinary skill in the art. Specifically, the medical devices 10 include any devices that are used, at least in part, to penetrate the body of a patient. Non-limiting examples of medical devices 10 include lumen-supporting devices (e.g., stents), catheters, guide wires, balloons, filters (e.g., vena cava filters), subcutaneous infusion devices, biosensors, stent grafts, vascular grafts, hernia grafts, intraluminal paving systems, soft tissue and hard tissue implants such as orthopedic plates and rods, joint implants, tooth and jaw implants, intramedulary implants, biologic scaffolding, metallic alloy ligatures, vascular access ports, artificial heart housings, heart valve struts and stents (used in support of biologic heart valves), aneurysm filling coils and other coiled coil devices, trans myocardial revascularization (“TMR”) devices, percutaneous myocardial revascularization (“PMR”) devices, hypodermic needles, soft tissue clips, staples, screws, holding or fastening devices, other types of medically useful needles and closures, organ or tissue transplant interfaces, and devices used in connection with drug-delivery. Such medical devices 10 can be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like. Any exposed surface of these medical devices 10 can comprise the biocompatible nanostructured ceramic material 12 disclosed herein.

By way of an exemplary embodiment, the medical device 10 is a lumen-supporting device, such as a stent. The biocompatible nanostructured ceramic material 12 of the lumen-supporting device can have an average grain size dimension of about 1 nm to about 1000 nm, a strain to failure of at least about 1 percent, and a cross-sectional hardness greater than or equal to about 350 kg/mm² as described above. If the lumen-supporting device does not require mechanical deformation or expansion, the biocompatible nanostructured ceramic material 12 can be in the form of a free standing bulk member, as illustrated in FIGS. 1 and 2. Depending on the extent to which the lumen-supporting device can be deformed after implantation, such as by using a balloon catheter, the lumen-supporting device can comprise a structural member 14 such as those shown in FIGS. 3 and 4, onto which the biocompatible nanostructured ceramic material 12 is disposed. The structural member 14 can have a solid or mesh-like structure made of a deformable or elastically malleable material. Exemplary materials used to construct the structural member 14 for the lumen-supporting device include, but are not limited to, stainless steel, a shape memory nickel-titanium alloy, non-ferrous metals, and bioabsorbable or biodegradable polymers.

It should be recognized that different biocompatible nanostructured ceramic materials 12 can be used on different portions of the structural element 14 of the lumen-supporting device. For example, the coating of the interior (abluminal surface of the structural element 14) of the lumen-supporting device can be different from the exterior (luminal) biocompatible nanostructured ceramic material 12 coating.

The lumen-supporting device can be implanted into a variety of lumina, including but not limited to vascular, cerebral, urethral, ureteral, biliary, tracheal, brachial, gastrointestinal, and esophageal lumina.

If it is desirable for the lumen-supporting device to also function as a drug delivery device (e.g., to treat ailments such as renal calculi, vascular stenosis, coronary artery disease, femoral artery occlusion, iliac artery occlusion, peripheral vascular disease, carotid stenosis, and the like) or assist in tissue engineering for regrowth of organs, the lumen-supporting device can also include the optional biologically active agent, which might or might not be combined with a polymeric material as a carrier.

In another exemplary embodiment, the medical device 10 is a fastening device such as a staple or clip. Since the fastening device can undergo significant deformation, it generally comprises a structural member 14, made of a deformable or elastically malleable material, onto which the biocompatible nanostructured ceramic material 12 is disposed. Exemplary materials used to construct the structural member 14 for the fastening device include, but are not limited to, stainless steel, a shape memory nickel-titanium alloy, non-ferrous metals, and bioabsorbable or biodegradable polymers. Also, because of the significant deformation that can be experienced by the fastening device, the coating of the biocompatible nanostructured ceramic material 12 can have-an increased strain to failure. If it is desirable for the fastening device to assist in preventing infections from a surgical ligation, it can also include the optional biologically active agent, which might or might not be combined with a polymeric material as a carrier.,

In yet another exemplary embodiment, the medical device 10 is a hernia or vascular graft. Similar to the lumen-supporting device, the biocompatible nanostructured ceramic material 12 of the graft can be a free standing bulk member or a coating on a structural member 14 (e.g., a woven mesh-like structure). Exemplary materials used to construct the structural member 14 for the graft include so-called “implant-grade” non-biodegradable polymers, biodegradable polymers, and biologic scaffolding materials. The graft can also include the optional biologically active agent to treat or prevent graft occlusion, graft infection, anastomotic aneurism (vascular graft), distal embolism (vascular graft), lower fossa abscesses (hernia graft)., or the like.

The disclosure is further illustrated by the following non-limiting examples.

### EXAMPLE 1

### Formation of a Dense Composite Oxide Layer via Air Plasma Spray

A composite of spray dried powder spheres having an overall composition of 13 wt % TiO₂, 13 wt % Y₂O₃, 10 wt % ZrO₂, 6 wt % CeO₂, and the balance of Al₂O₃ (commercially available from Inframat Corp. under the tradename of NANOX S2613), was used as a feedstock. The feedstock was plasma thermal sprayed. using a Metco 9MB plasma spray system (all Metco products mentioned herein are sold by Sulzer Metco Ltd.), onto a metal substrate which had been sandblasted using alumina granules prior to thermal spraying. A mixture of argon and hydrogen gases was used in conjunction with a GH-type nozzle (Metco) to generate a hot and high-velocity plasma flame. The powder-feeding rate was between about 1.5 to about 2.0 pounds per hour (lb/hr), which corresponded to a deposition rate of about 50 to about 120 micrometers (μm) per pass. The substrate was preheated to a temperature of about 120 degrees Celsius (° C.), which was maintained during the spray process when a small standoff distance and low gun traverse speed were selected. Representative plasma spraying parameters for the dense composite oxide layer were as follows:

Plasma gases:
Primary gas: Argon (100 pounds per square inch (PSI), 80 standard cubic feet per hour (SCFH))
Secondary gas: H₂ (50 PSI)

Plasma power: 45.5 kilowatts (KW) (650 Amperes (A)/70 volts (V))

Standoff distance: 3.5 inches

Gun speed:
Traverse speed: 500 to 600 millimeters per second (mm/s)
Vertical speed: 6 mm/s

Powder feed rate: 1.5-2.0 lb/hr

Substrate temperature:
Preheating: 100-120° C.
During spraying: 120-150° C.

The various plasma sprayed layers of the composite oxide had densities greater than about 98% of the theoretical density, and thicknesses greater than or equal to about 50 μm. A well-bonded interface between the coatings and the substrates was observed using scanning electron microscopy.

### EXAMPLE 2

### Formation of a Dense Al₂O₃ Layer via Air Plasma Spray

Angular, fused, and crushed Al₂O₃ powder (Metco 105SFP) was used as a feedstock. The feedstock was plasma thermal sprayed using a Metco 9MB plasma spray system, onto a metal substrate which had been sandblasted using alumina granules prior to thermal spraying. A mixture of argon and hydrogen gases was used in conjunction with a GP-type nozzle (Metco) to generate a hot and high-velocity plasma flame. The powder-feeding rate was between about 2.0 to about 2.5 lb/hr, which corresponded to a deposition rate of about 50 to about 120 μm per pass. The substrate was preheated to a temperature of about 120° C., which was maintained during the spray process when a small standoff distance and low gun traverse speed were selected. Representative plasma spraying parameters for the dense Al₂O₃ layer were as follows:

Plasma gases:
Primary gas: Argon (100 PSI, 100 SCFH)
Secondary gas: H₂, (50 PSI)

Plasma power: 42 KW (600 A/70 V)

Standoff distance: 3.5 inches

Gun speed:
Traverse speed: 1000 mm/s
Vertical speed: 8 mm/s

Powder feed rate: 2.0-2.5 lb/hr

Substrate temperature:
Preheating: 100-120° C.
During spraying: 120-150° C.

The various plasma sprayed layers of Al₂O₃ had densities greater than about 98% and thicknesses greater than or equal to about 30 μm. A well-bonded interface between the coatings and the substrates was observed using scanning electron microscopy.

### EXAMPLE 3

### Formation of a Dense Composite Oxide Layer via Air Plasma Spray

A composite of spray dried powder spheres having an overall composition of Cr₂O₃-5SiO₂-3TiO₂ (Metco 136F) was used as a feedstock. The feedstock was plasma thermal sprayed, using a Metco 9MB plasma spray system, onto a metal substrate which had been sandblasted using alumina granules prior to thermal spraying. A mixture of argon and hydrogen gases was used in conjunction with a GH-type nozzle (Metco) to generate a hot and high-velocity plasma flame. The powder-feeding rate was between about 2.5 to about 3.0 lb/hr, which corresponded to a deposition rate of about 15 to about 30 μm per pass. The substrate was preheated to a temperature of about 120° C., which was maintained during the spray process when a small standoff distance and low gun traverse speed were selected. A cross-cooling jet was used to cool the substrate with an air flow at about 40 PSI. Representative plasma spraying parameters for the dense composite oxide layer were as follows:

Plasma gases:
Primary gas: Argon (100 PSI, 80 SCFH)
Secondary gas: H₂, (50 PSI)

Plasma power: 42 KW (600 A/70 V)

Standoff distance: 2.5 inches

Gun speed:
Traverse speed: 1000 mm/s
Vertical speed: 8 mm/s

Powder feed rate: 2.5-3.0 lb/hr

Substrate temperature:
Preheating: 100-120° C
During spraying: 120-150° C.

The various plasma sprayed layers of the composite oxide had densities of greater than about 98%, and thicknesses greater than or equal to about 20 μm. A well-bonded interface between the coatings and the substrates was observed using scanning electron microscopy.

### EXAMPLE 4

### Formation of Porous ZrO₂-8 wt % Y₂O₃ Layer via Air Plasma Spray

Densified spheres having a composition of ZrO₂-8 wt % Y₂O₃ (Metco 204NS) was used as a feedstock. The feedstock was plasma thermal sprayed, using a Metco 9MB plasma spray system, onto a metal substrate which had been sandblasted using alumina granules prior to thermal spraying. A mixture of argon and hydrogen gases was used in conjunction with a GH-type nozzle (Metco) to generate a hot and high-velocity plasma flame. The powder-feeding rate was between about 5.5 to about 6.0 lb/hr, which corresponded to a deposition rate of about 50 to about 60 μm per pass. The substrate was preheated to a temperature of about 120° C., which was maintained during the spray process when a small standoff distance and low gun traverse speed were selected. Representative plasma spraying parameters for the porous ZrO₂-8 wt % Y₂O₃ layer were as follows:

Plasma gases:
Primary gas: Argon (100 PSI, 80 SCFH)
Secondary gas: H₂, (50 PSI)

Plasma power: 39 KW (600 A/65 V)

Standoff distance: 2.5 inches

Gun speed:
Traverse speed: 500 mm/s
Vertical speed: 8 mm/s

Powder feed rate: 5.5-6.0 lb/hr

Substrate temperature:
Preheating: 100-120° C.
During spraying: 120-150° C.

The various plasma sprayed layers of ZrO₂-8 wt % Y₂O₃ had porosities of about 15 to about 20%, and thicknesses greater than or equal to about 50 μm. The primary phase in the coatings was tetragonal, as determined by powder X-ray diffraction. A well-bonded interface between the coatings and the substrates was observed using scanning electron microscopy.

### EXAMPLE 5

### Formation of a Porous Al₂O₃ Layer via Solution Plasma Spray

An aqueous solution made from an aluminum salt was used as a feedstock. A liquid delivery system equipped with reservoirs, flow-rate regulators, and an atomizing liquid injector, was used to deliver the solution to a plasma heating source at a constant flow rate. The feedstock was plasma thermal sprayed, using a Metco 9MB plasma spray system, onto a metal substrate which had been sandblasted using alumina granules prior to thermal spraying. A mixture of argon and hydrogen gases was used in conjunction with a GP-type nozzle (Metco) to generate a hot and high-velocity plasma flame. The solution feeding rate was between about 50 and about 80 milliliters per minute (ml/min), which corresponded to a deposition rate of about 10 to about 20 μm per pass. The substrate was preheated to a temperature of about 250° C., which was maintained during the spray process when a small standoff distance and low gun traverse speed were selected. Representative plasma spraying parameters for the porous Al₂O₃ layer were as follows:

Plasma gases:
Primary gas: Argon (100 PSI, 140 SCFH)
Secondary gas: H₂, (50 PSI)

Plasma power: 39 KW (600 A/65 V)

Standoff distance: 2 inches

Gun speed:
Traverse speed: 1000 mm/s
Vertical speed: 4 mm/s

Solution feed rate: 50-80 milliliter/minute (ml/min)

Substrate temperature:
Preheating: >250° C.
During spraying: 250-350° C.

The various plasma sprayed layers of Al₂O₃ had porosities of about 30 to about 40% and thicknesses greater than or equal to about 10 μm.

### EXAMPLE 6

### Formation of a Porous ZrO₂-8 wt % Y₂O₃ Layer via Solution Plasma Spray

An aqueous solution of ZrO₂-8 wt % Y₂O₃ was used as a feedstock. A liquid delivery system equipped with reservoirs, flow-rate regulators, and an atomizing liquid injector, was used to deliver the solution to a plasma heating source at a constant flow rate. The feedstock was plasma thermal sprayed, using a Metco 9MB plasma spray system, onto a metal substrate which had been sandblasted using alumina granules prior to thermal spraying. A mixture of argon and hydrogen gases was used in conjunction with a GP-type nozzle (Metco) to generate a hot and high-velocity plasma flame. The solution feeding rate was between about 20 to about 30 ml/min, which corresponded to a deposition rate of about 5 to about 15 μm per pass. The substrate was preheated to a temperature of about 250° C., which was maintained during the spray process when a small standoff distance and low gun traverse speed were selected. Representative plasma spraying parameters for the porous ZrO₂-8 wt % Y₂O₃ layer were as follows:

Plasma gases:
Primary gas: Argon (100 PSI, 140 SCFH)
Secondary gas: H₂, (50 PSI)

Plasma power: 45.5 KW (650 A/70 V)

Standoff distance: 2 inches

Gun speed:
Traverse speed: 1000 mm/s
Vertical speed: 4 mm/s

Solution feed rate: 20-30 ml/min

Substrate temperature:
Preheating: >250° C.
During spraying: 250-350° C.

The various plasma sprayed layers of ZrO₂-8 wt % Y₂O₃ had porosities of about 18 to about 22% and thicknesses greater than or equal to about 5 μm. The primary phase in the coatings was tetragonal, as determined by powder X-ray diffraction.

### EXAMPLE 7

### Formation of a Porous Al₂O₃/TiO₂ Layer via Solution Plasma Spray

An aqueous solution of Al₂O₃-5 mole percent (mol %) TiO₂, made from aluminum and titanium salts, was used as a feedstock. A liquid delivery system equipped with reservoirs, flow-rate regulators, and an atomizing liquid injector, was used to deliver the solution to a plasma heating source at a constant flow rate. The feedstock was plasma thermal sprayed, using a Metco 9MB plasma spray system, onto a metal substrate which had been sandblasted using alumina granules prior to thermal spraying. A mixture of argon and hydrogen gases was used in conjunction with a GP-type nozzle (Metco) to generate a hot and high-velocity plasma flame. The solution feeding rate was between about 30 to about 40 ml/min, which corresponded to a deposition rate of about 5 to about 15 μm per pass. The substrate was preheated to a temperature of about 250° C., which was maintained during the spray process when a small standoff distance and low gun traverse speed were selected. Representative plasma spraying parameters for the porous Al₂O₃-5 mol % TiO₂ layer were as follows:

Plasma gases:
Primary gas: Argon (100 PSI, 80 SCHF)
Secondary gas: H₂, (50 PSI)

Plasma power: 45.5 KW (650 A/70 V)

Standoff distance: 2 inches

Gun speed:
Traverse speed: 1000 mm/s
Vertical speed: 4 mm/s

Solution feed rate: 30 -40 ml/min

Substrate temperature:
Preheating: >250° C.
During spraying: 250-350° C.

The various plasma sprayed layers of Al₂O₃/TiO₂ had porosities of about 20 to about 30% and thicknesses greater than or equal to about 10 μm.

### EXAMPLE 8

### Formation of a Composite Oxide Layer via Solution Plasma Spray

An aqueous solution of 6 mol % Y₂O₃, 20 mol % Al₂O₃, 5 mol % TiO₂, and the balance of ZrO₂, which were made from zirconium, yttrium, aluminum and titanium salts, was used as a feedstock. A liquid delivery system equipped with reservoirs, flow-rate regulators, and an atomizing liquid injector, was used to deliver the solution to a plasma heating source at a constant flow rate. The feedstock was plasma thermal sprayed, using a Metco 9MB plasma spray system, onto a metal substrate which had been sandblasted using alumina granules prior to thermal spraying. A mixture of argon and hydrogen gases was used in conjunction with a GP-type nozzle (Metco) to generate a hot and high-velocity plasma flame. Tie solution feeding rate was between about 20 to about 25 ml/min, which corresponded to a deposition rate of about 5 to about 10 μm per pass. The substrate was preheated to a temperature of about 250° C., which was maintained during the spray process when a small standoff distance and low gun traverse speed were selected. Representative plasma spraying parameters for the porous composite oxide layer were as follows:

Plasma gases:
Primary gas: Argon (100 PSI, 140 SCFH)
Secondary gas: H₂, (50 PSI)

Plasma power: 45.5 KW (650 A/70 V)

Standoff distance: 2 inches

Gun speed:
Traverse speed: 1000 mm/s
Vertical speed: 4 mm/s

Solution feed rate: 20-25 ml/min

Substrate temperature:
Preheating: >250° C.
During spraying: 250-450° C.

The various plasma sprayed layers of the composite oxide had porosities of about 18 to about 22% and thicknesses greater than or equal to about 10 μm.

### EXAMPLE 9

### Formation of a TiO₂ Layer via Slurry Plasma Spray

A 300 grams per liter (g/l) slurry of TiO₂, made from mixing fine (about 10 to about 20 nm) TiO₂ particles and water, was used as feedstock. A liquid delivery system equipped with reservoirs, flow-rate regulators, and an atomizing liquid injector, was used to deliver the solution to a plasma heating source at a constant flow rate. The feedstock was plasma thermal sprayed, using a Metco 9MB plasma spray system, onto a metal substrate which had been sandblasted using alumina granules prior to thermal spraying. A mixture of argon and hydrogen gases was used in conjunction with a GP-type nozzle (Metco) to generate a hot and high-velocity plasma flame. The solution feeding rate was between about 30 to about 40 ml/min, which corresponded to a deposition rate of about 10 to about 20 μm per pass. The substrate was preheated to a temperature of about 150° C., which was maintained during the spray process when a small standoff distance and low gun traverse speed were selected. Representative plasma spraying parameters for the porous TiO₂ layer were as follows:

Plasma gases:
Primary gas: Argon (100 PSI, 80 SCFH)
Secondary gas: H₂, (50 PSI)

Plasma power: 39 KW (600 A/65 V)

Standoff distance: 2 inches

Gun speed:
Traverse speed: 1000 mm/s
Vertical speed: 4 min/s

Solution feed rate: 30-40 ml/min

Substrate temperature:
Preheating: >150° C.
During spraying: 150-250° C.

The various plasma sprayed layers of TiO₂ had porosities of about 5 to about 25% and thicknesses greater than or equal to about 10 μm.

### EXAMPLE 10

### Formation of a Dense, Bulk, Composite Oxide Material via Air Plasma Spray

A composite of spray dried powder spheres having an overall composition of 13 wt % TiO₂, 13 wt % Y₂O₃, 10 wt % ZrO₂, 6 wt % CeO₂, and the balance of NANOX S2613 Al₂O₃ was used as a feedstock. The feedstock was plasma thermal sprayed, using a Metco 9MB plasma spray system, onto a metal substrate which had been sandblasted using 180 grit alumina granules prior to thermal spraying. A mixture of argon and hydrogen gases was used in conjunction with a GH-type nozzle (Metco) to generate a hot and high-velocity plasma flame. The powder-feeding rate was between about 1.5 to about 2.0 lb/hr, which corresponded to a deposition rate of about 50 to about 120 μm per pass. The substrate was preheated to a temperature of about 120° C., which was maintained during the spray process when a small standoff distance and low gun traverse speed were selected. Representative plasma spraying parameters for the dense composite oxide layer were as follows:

Plasma gases:
Primary gas: Argon (100 PSI, 80 SCFH)
Secondary gas: H₂, (50 PSI)

Plasma power: 45.5 KW (650 A/70 V)

Standoff distance: 3.5 inches

Gun speed:
Traverse speed: 500-600 mm/s
Vertical speed: 6 mm/s

Powder feed rate: 1.5-2.0 lb/hr

Substrate temperature:
Preheating: 100-120° C.
During spraying: 120-150° C.

After plasma spraying the composite oxide layer onto the metal substrate, the substrate was removed. The various free-standing bulk composite oxide members had densities of greater than or equal to about 98% and thicknesses of about 500 μm to about 3 mm.

### EXAMPLE 11

### Formation of a Porous, Bulk ZrO₂-8 wt % Y₂O₃ Material via Solution Plasma Spray

aqueous solution of ZrO₂-8 wt % Y₂O₃ was used as a feedstock. A liquid delivery system equipped with reservoirs, flow-rate regulators, and an atomizing liquid injector, was used to deliver the solution to a plasma heating source at a constant flow rate. The feedstock was plasma thermal sprayed, using a Metco 9MB plasma spray system, onto a metal substrate which had been sandblasted using alumina granules prior to thermal spraying. A mixture of argon and hydrogen gases was used in conjunction with a GP-type nozzle (Metco) to generate a hot and high-velocity plasma flame. The solution feeding rate was between about 20 to about 30 ml/min, which corresponded to a deposition rate of about 5 to about 15 μm per pass. The substrate was preheated to a temperature of about 250° C., which was maintained during the spray process when a small standoff distance and low gun traverse speed were selected. Representative plasma spraying parameters for the porous ZrO₂-8 wt % Y₂O₃ layer were as follows:

Plasma gases:
Primary gas: Argon (100 PSI, 140 SCFH)
Secondary gas: H₂, (50 PSI)

Plasma power: 45.5 KW (650 A/70 V)

Standoff distance: 2 inches

Gun speed:
Traverse speed: 1000 mm/s
Vertical speed: 4 mm/s

Solution feed rate: 20-30 ml/min

Substrate temperature:
Preheating: >250° C.
During spraying: 250-350° C.

After plasma spraying the ZrO₂-8 wt % Y₂O₃ layer onto the metal substrate, the substrate was removed. The various flee-standing bulk ZrO₂-8 wt % Y₂O₃ members had porosities of about 18 to about 22% and thicknesses of about 500 μm to about 4.0 mm.

### EXAMPLE 12

### Formation of a Gradient Composite Layer via Air Plasma Spray

Various mixtures of a composite of spray dried powder spheres having an overall composition of 13 wt % TiO₂, 13 wt % Y₂O₃, 10 wt % ZrO₂, 6 wt % CeO₂, and the balance of NANOX S2613 Al₂O₃ and Fe₃O₄ were used as a f(eedstock. Individual samples of the composite were made having 0, 25, 50, and 75 wt % Fe₃O₄. The feedstock was plasma thermal sprayed, using a Metco 9MB plasma spray system, onto a metal substrate which had been sandblasted using alumina granules prior to thermal spraying. A mixture of argon and hydrogen gases was used in conjunction with a GH-type nozzle (Metco) to generate a hot and high-velocity plasma name. The powder-feeding rate was between about 2.0 to about 2.5 lb/hr, which corresponded to a deposition rate of about 50 to about 120 μm per pass. A gradient in the coating was produced by independently and sequentially spraying die 0, 25, 50, and 75 wt % FeCO₄ feedstock mixtures. The substrate was preheated to a temperature of about 120° C., which was maintained during the spray process when a small standoff distance and low gun traverse speed were selected. Representative plasma spraying parameters for the dense composite oxide layer were as follows:

Plasma gases:
Primary gas: Argon (100 PSI, 80 SCFH)
Secondary gas: H₂, (50 PSI)

Plasma power: 45.5 KW (650 A/70 V)

Standoff distance: 3.5-4 inches

Gun speed:
Traverse speed: 500-600 mm/s
Vertical speed: 6 mm/s

Powder feed rate: 2.0-2.5 lb/hr

Substrate temperature:
Preheating: 100-120° C.
During spraying: 120-150° C.

The various composite layers with gradients had densities of greater than or equal to about 98%.

As used herein, the terms “a” and “an” do not denote a limitation of quantity, but rather denote the presence of at least one of the referenced items. Moreover, the endpoints of all ranges directed to the same component or property are inclusive of the endpoint and independently combinable,(e.g., “about 5 wt % to about 20 wt %,” is inclusive of the endpoints and all intermediate values of the ranges of about 5 wt % to about 20 wt %). Reference throughout the specification to “one embodiment”, “another embodiment”, “an embodiment”, and so forth means that a particular element (e.g., feature, structure, and/or characteristic) described in connection with the embodiment is included in at least one embodiment described herein, and might or might not be present in other embodiments. In addition, it is to be understood that the described elements may be combined in any suitable manner in the various embodiments. Unless defined otherwise, technical and scientific terms used herein have the same meaning as is commonly understood by one of skill in the art to which this invention belongs.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

1. A medical device, comprising a biocompatible nanostructured ceramic material having an average grain size dimension of about 1 nanometer to about 1000 nanometers, a strain to failure of at least about 1 percent, and a cross-sectional hardness greater than or equal to about 350 kilograms per square millimeter.

2. The medical device of claim 1, wherein the biocompatible nanostructured ceramic material is a film disposed on a surface of a structural member of the medical device.

3. The medical device of claim 2, wherein the structural member comprises a metal, alloy, polymer, biologic scaffolding, or a combination comprising at least one of the foregoing.

4. The medical device of claim 1, wherein the biocompatible nanostructured ceramic material and a tissue adherent material or a metal layer are disposed on opposing surfaces of a structural member of the medical device.

5. The medical device of claim 1, wherein the biocompatible nanostructured ceramic material and a tissue adherent material are disposed on different portions of a surface of a structural member of the medical device.

6. The medical device of claim 1, wherein the biocompatible nanostructured ceramic material and a cathode are disposed on different portions of a first surface of a structural member of the medical device, and further comprising a positively charged biologically active agent disposed underneath a second surface of the structural member opposite from the first surface and an anode disposed underneath the biologically active agent for causing the biologically active agent to pass through the ceramic material.

7. The medical device of claim 1, wherein the biocompatible nanostructured ceramic material is a free standing bulk member.

8. The medical device of claim 1, further comprising a biologically active agent.

9. The medical device of claim 8, wherein the biologically active agent is disposed within a pore of the biocompatible nanostructured ceramic material, upon the biocompatible nanostructured ceramic material, underneath the biocompatible nanostructured ceramic material, on an opposite side of a structural member from the biocompatible nanostructured ceramic material, or a combination comprising at least one of the foregoing.

10. The medical device of claim 1, wherein the biocompatible nanostructured ceramic material has a thickness greater than or equal to about 1 micrometer.

11. The medical device of claim 1, wherein the biocompatible nanostructured ceramic material has a density of greater than or equal to about 90 percent of a theoretical density of the biocompatible nanostructured ceramic material.

12. The medical device of claim 1, wherein the biocompatible nanostructured ceramic material has a porosity of greater than or equal to about 10 percent of a total volume of the biocompatible nanostructured ceramic material.

13. The medical device of claim 1, wherein an average longest dimension of a pore within the biocompatible nanostructured ceramic material is less than or equal to about 1 micrometer.

14. The medical device of claim 1, wherein the medical device is a catheter, guide wire, balloon, filter, subcutaneous infusion device, biosensor, stent graft, vascular graft, hernia graft, intraluminal paving system, soft tissue implant, hard tissue implant, intramedulary implant, biologic scaffolding, ligature, vascular access port, artificial heart housing, heart valve strut, aneurysm filling coil, trans myocardial revascularization device, percutaneous myocardial revascularization device, hypodermic needle, soft tissue clip, staple, screw, holding device, fastening device, organ transplant interface, tissue transplant interface, or drug delivery device.

15. A medical device comprising:
a structural member comprising a metal, an alloy, a polymer, a biologic scaffolding, or a combination comprising at least one of the foregoing; and
a film comprising a biocompatible nanostructured ceramic material at least partially coating a surface of the structural member, the film having a thickness greater than or equal to about 1 micrometer, an average grain size dimension of about 1 nanometer to about 1000 nanometers, a strain to failure of at least about 1 percent, and a cross-sectional hardness greater than or equal to about 350 kilograms per square millimeter.

16. The medical device of claim 15, wherein the biocompatible nanostructured ceramic material has a density of greater than or equal to about 90 percent of a theoretical density of the biocompatible nanostructured ceramic material.

17. The medical device of claim 15, wherein the biocompatible nanostructured ceramic material has a porosity of greater than or equal to about 10 percent of a total volume of the biocompatible nanostructured ceramic material.

18. The medical device of claim 15, wherein an average longest dimension of a pore within the biocompatible nanostructured ceramic material is less than or equal to about 1 micrometer.

19. The medical device of claim 15, wherein the medical device is a catheter, guide wire, balloon, filter, subcutaneous infusion device, biosensor, stent graft, vascular graft, hernia graft, intraluminal paving system, soft tissue implant, hard tissue implant, intramedulary implant, biologic scaffolding, ligature, vascular access port, artificial heart housing, heart valve strut, aneurysm filling coil, trans myocardial revascularization device, percutaneous myocardial revascularization device, hypodermic needle, soft tissue clip, staple, screw, holding device, fastening device, organ transplant interface, tissue transplant interface, or drug delivery device.

20. A method comprising: surgically implanting a medical device, comprising a biocompatible nanostructured ceramic material having an average grain size dimension of about 1 nanometer to about 1000 nanometers, a strain to failure of at least about 1 percent, and a cross-sectional hardness greater than or equal to about 350 kilograms per square millimeter.

21. The method of claim 20, wherein surgically implanting the medical device comprises surgically implanting the medical device in a coronary vasculatire, esophagus, trachea, colon, biliary tract, urinary tract, prostate, brain, lung, liver, heart, skeletal muscle, kidney, bladder, intestine, stomach, pancreas, ovary, cartilage, eye, or bone.

22. The method of claim 20, wherein the biocompatible nanostructured ceramic material has a density of greater than or equal to about 90 percent of a theoretical density of the biocompatible nanostructured ceramic material.

23. The method of claim 20, wherein the biocompatible nanostructured ceramic material has a porosity of greater than or equal to about 10 percent of a total volume of the biocompatible nanostructured ceramic material.

24. The method of claim 20, wherein an average longest dimension of a pore within the biocompatible nanostructured ceramic material is less than or equal to about 1 micrometer.

25. A method of making a medical device, comprising: consolidating a biocompatible nanoparticulate ceramic powder into a free standing bulk biocompatible ceramic nanostructured ceramic material having an average grain size dimension of about 1 nanometer to about 1000 nanometers, a strain to failure of at least about 1 percent, and a cross-sectional hardness greater than or equal to about 350 kilograms per square millimeter.

26. The method of claim 25, further comprising shaping the free standing bulk biocompatible ceramic nanostructured ceramic material.

27. The method of claim 25, further comprising disposing a biologically active agent on the free standing bulk biocompatible ceramic nanostructured ceramic material, within a pore of the free standing bulk biocompatible ceramic nanostructured ceramic material, or a combination comprising at least one of the foregoing.

28. The method of claim 25, further comprising annealing, grinding, or polishing the free standing bulk biocompatible ceramic nanostructured ceramic material.

29. A method of making a medical device, comprising: disposing a coating of a biocompatible nanostructured ceramic material having an average grain size dimension of about 1 nanometer to about 1000 nanometers, a strain to failure of at least about 1 percent, and a cross-sectional hardness greater than or equal to about 350 kilograms per square millimeter onto at least a portion of a surface of a structural member of the medical device.

30. The method of claim 29, further comprising disposing a biologically active agent directly on the coating of the biocompatible ceramic nanostructured ceramic material, between the coating of the biocompatible ceramic nanostructured ceramic material and the structural member, within a pore of the coating of the biocompatible ceramic nanostructured material, on an opposite side of the structural member from the coating of the biocompatible ceramic nanostructured ceramic material, or a combination comprising at least one of the foregoing.

31. The method of claim 29, wherein disposing the coating of the biocompatible nanostructured ceramic material comprises thermal spraying, chemical vapor deposition, physical vapor deposition, sputtering, ion plating, cathodic arc deposition, atomic layer epitaxy, molecular beam epitaxy, powder sintering, electrophoresis, electroplating, injection molding, or a combination comprising at least one of the foregoing.

32. The method of claim 29, further comprising annealing, grinding, or polishing the coating of the biocompatible nanostructured ceramic material.

33. The method of claim 29, further comprising disposing a tissue adherent material on the surface of the structural member adjacent to the coating of the biocompatible nanostructured ceramic material.

34. The method of claim 29, further comprising: disposing an anode on the surface of the structural member adjacent to the coating of the biocompatible nanostructured ceramic material; disposing a biologically active agent on an opposite side of the structural member from the coating of the biocompatible ceramic nanostructured ceramic material; and disposing a cathode underneath tile biologically active agent.
